(19) United States
(12) Patent Application Publication
(10) Pub. No.: US 2009/0054777 A1
(43) Pub. Date: Feb. 26, 2009

(54) ULTRASONIC DIAGNOSTIC APPARATUS

(76) Inventors: Shinichi Amemiya, Tokyo (JP); Dai Morita, Tokyo (JP); Koji Yanagihara, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/196,811

(22) Filed: Aug. 22, 2008

(30) Foreign Application Priority Data
Aug. 24, 2007 (JP) 2007-218282

(51) Int. Cl. A61B 8/00

(52) U.S. Cl. 600/443

(57) ABSTRACT

An ultrasonic diagnostic apparatus includes: a plurality of transmitter-receiver units each having a probe connector, a transmitter-receiver and a power supply; a plurality of display units each having an image display and a power supply; a plurality of operation units each having an operating part and a power supply; and a control unit. The plural transmitter-receiver units, the plural display units and the plural operation units can be connected to the control unit, and the control unit can control the thus-connected plural transmitter-receiver units, plural display units and plural operation units each independently.

### CROSS REFERENCE TO RELATED APPLICATIONS

This application claims the benefit of Japanese Patent Application No. 2007-218282 filed Aug. 24, 2007, which is hereby incorporated by reference in its entirety.

### BACKGROUND OF THE INVENTION

The subject matter disclosed herein relates to an ultrasonic diagnostic apparatus and more particularly to an ultrasonic diagnostic apparatus in which information and resources can be shared by plural operators and which is high in the degree of freedom of configuration in actual operation.

Heretofore there has been known an ultrasonic diagnostic apparatus of a configuration in which plural clients are connected to one server (see, for example, Japanese Unexamined Patent Publication No. 2006-187593).

In the above conventional ultrasonic diagnostic apparatus, since plural clients are connected to one server, it is possible to share information and resources.

On the clients' side, however, there is used a configuration including a transmitter-receiver (beam former), a display and an operating part as a set, and thus there has been the problem that the degree of freedom of configuration in actual operation is low. For example, when a sonographer moves from a certain ultrasonographic room to another ultrasonographic room, if he or she wants to use his or her well-used operating part, it is necessary to move the set of transmitter-receiver, display and operating part to the ultrasonographic room to which he or she is going.

### BRIEF DESCRIPTION OF THE INVENTION

It is desirable that the problem described previously is solved.

In a first aspect of the invention there is provided an ultrasonic diagnostic apparatus including a plurality of transmitter-receiver units each having a probe connector, a transmitter-receiver and a power supply, a plurality of display units each having an image display and a power supply, a plurality of operation units each having an operating part and a power supply, and a control unit, wherein the plural transmitter-receiver units, the plural display units and the plural operation units can be connected to the control unit, and the control unit can control the thus-connected plural transmitter-receiver units, plural display units and plural operation units each independently.

By the connection referred to in the above configuration it is meant to include both direct connection and indirect connection via another unit(s) by wire or wireless.

According to the ultrasonic diagnostic apparatus in the above first aspect, since the plural transmitter-receiver units, plural display units and plural operation units are connected to one control unit, it is possible to share information and resources. The transmitter-receiver units, display units and operation units are each independent also in power supply, thus permitting free selection and combination. Consequently, the degree of freedom of configuration in actual operation is high. For example, when a sonographer moves from a certain ultrasonographic room to another ultrasonographic room, imaging parameters recorded in the control unit can be utilized also in the ultrasonographic room to which the sonographer moves. Besides, it becomes possible for the sonographer to carry only his or her well-used operation unit to the ultrasonographic room to which he or she is going.

In a second aspect of the invention there is provided, in combination with the above first aspect, an ultrasonic diagnostic apparatus wherein the transmitter-receiver units, the display units and the operation units can be connected directly to the control unit.

According to the ultrasonic diagnostic apparatus in the above second aspect, it suffices to provide an interface for connecting the transmitter-receiver units, the display units and the operation units directly to the control unit. Thus, it is possible to simplify the configuration.

In a third aspect of the invention there is provided, in combination with the above first aspect, an ultrasonic diagnostic apparatus wherein any one type of the units selected from among the transmitter-receiver units, the display units and the operation units can be connected directly to the control unit and the other two types of the units can be connected directly to the one type of the units and can be connected indirectly to the control unit through the one type of the units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above third aspect, only any one type of the units selected from among the transmitter-receiver units, the display units and the operation units are connected directly to the control unit, so that a small number of communication lines suffice between the ultrasonographic rooms and the control room.

In a fourth aspect of the invention there is provided, in combination with the above third aspect, an ultrasonic diagnostic apparatus wherein the transmitter-receiver units can be connected directly to the control unit, and the display units and the operation units can be connected directly to the transmitter-receiver units and can be connected indirectly to the control unit through the transmitter-receiver units.

For example, when one transmitter-receiver unit, one display unit and one operation unit are installed in each of plural ultrasonographic rooms and the control unit is installed in a control room, communication lines needed between the ultrasonographic rooms and the control room. However, according to the ultrasonic diagnostic apparatus in the above fourth aspect, only the transmitter-receiver units are connected directly to the control unit, a small number of communication lines suffice between the ultrasonographic rooms and the control room.

In a fifth aspect of the invention there is provided, in combination with the above fourth aspect, an ultrasonic diagnostic apparatus wherein at least one of the display units and at least one of the operation units can be connected directly to the control unit.

According to the ultrasonic diagnostic apparatus in the above fifth aspect it is possible to adopt, for example, a mode of use such that a sonographer positioned near a patient uses a display unit and an operation unit both connected to a transmitter-receiver unit and a doctor positioned somewhat away from the patient uses a display unit and an operation unit both connected directly to the control unit. Thus, this ultrasonic diagnostic apparatus is useful for the doctor to train the sonographer practically.

In a sixth aspect of the invention there is provided, in combination with the above first aspect, an ultrasonic diagnostic apparatus wherein any two types of the units selected from among the transmitter-receiver units, the display units and the operation units can be connected directly to the control unit, and the other type of the units can be connected directly to any one of the two types of the units and can be connected indirectly to the control unit through any one of the two types of the units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above sixth aspect, only any two types of the units selected from among the transmitter-receiver units, the display units and the operation units can be connected directly to the control unit, so that a small number of communication lines suffice between the ultrasonographic rooms and the control room.

In a seventh aspect of the invention there is provided, in combination with the above sixth aspect, an ultrasonic diagnostic apparatus wherein the transmitter-receiver units and the operation units can be connected directly to the control unit, and the display units can be connected directly to the transmitter-receiver units and can be connected indirectly to the control unit through the transmitter-receiver units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above seventh aspect, only the transmitter-receiver units and the operation units are connected directly to the control unit, so that a small number of communication lines suffice between the ultrasonographic rooms and the control room.

In an eighth aspect of the invention there is provided, in combination with the above seventh aspect, an ultrasonic diagnostic apparatus wherein at least one of the display units can be connected directly to the control unit.

According to the ultrasonic diagnostic apparatus in the above eighth aspect it is possible to adopt, for example, a mode of use such that a sonographer positioned near a patient uses a display unit connected to a transmitter-receiver unit and a doctor positioned someway away from the patient uses a display unit connected directly to the control unit. This is useful for the doctor to train the sonographer practically.

In a ninth aspect of the invention there is provided, in combination with the above sixth aspect, an ultrasonic diagnostic apparatus wherein the transmitter-receiver units and the display units can be connected directly to the control unit, and the display units can be connected directly to the transmitter-receiver units and can be connected indirectly to the control unit through the transmitter-receiver units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above ninth aspect, only the transmitter-receiver units and the display units are connected directly to the control unit, so that a small number of communication lines suffice between the ultrasonographic rooms and the control room.

In a tenth aspect of the invention there is provided, in combination with the above ninth aspect, an ultrasonic diagnostic apparatus wherein at least one of the operation units can be connected directly to the control unit.

According to the ultrasonic diagnostic apparatus in the above tenth aspect it is possible to adopt, for example, a mode of use such that a sonographer positioned near a patient uses an operation unit connected to a transmitter-receiver unit and a doctor positioned somewhat away from the patient uses an operation unit connected directly to the control unit. This is useful for the doctor to train the sonographer practically.

In an eleventh aspect of the invention there is provided, in combination with the above sixth aspect, an ultrasonic diagnostic apparatus wherein the transmitter-receiver units and the operation units can be connected directly to the control unit, and the display units can be connected directly to the operation units and can be connected indirectly to the control unit through the operation units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above eleventh aspect, since only the transmitter-receiver units and the operation units are connected directly to the control unit, a small number of communication lines suffice between the ultrasonographic rooms and the control room.

In a twelfth aspect of the invention there is provided, in combination with the above eleventh aspect, an ultrasonic diagnostic apparatus wherein at least one of the display units can be connected directly to the control unit.

According to the ultrasonic diagnostic apparatus in the above twelfth aspect it is possible to adopt, for example, a mode of use such that a sonographer positioned near a patient uses a display unit connected to an operation unit and a doctor positioned somewhat away from the patient uses a display unit connected directly to the control unit. This is useful for the doctor to train the sonographer practically.

In a thirteenth aspect of the invention there is provided, in combination with the above sixth aspect, an ultrasonic diagnostic apparatus wherein the transmitter-receiver units and the display units can be connected directly to the control unit, and the operation units can be connected directly to the display units and can be connected indirectly to the control unit through the display unit.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above thirteenth aspect, since only the transmitter-receiver units and the display units are connected directly to the control unit, a small number of communication lines suffice between the ultrasonographic rooms and the control room.

In a fourteenth aspect of the invention there is provided, in combination with the above thirteenth aspect, an ultrasonic diagnostic apparatus wherein at least one of the operation units can be connected directly to the control unit.

According to the ultrasonic diagnostic apparatus in the above fourteenth aspect it is possible to adopt, for example, a mode of use such that a sonographer positioned near a patient uses an operation unit connected to a display unit and a doctor positioned somewhat away from the patient uses an operation unit connected directly to the control unit. This is useful for the doctor to train the sonographer practically.

In a fifteenth aspect of the invention there is provided, in combination with the above first aspect, an ultrasonic diagnostic apparatus wherein any one type of the units selected from among the transmitter-receiver units, the display units and the operation units can be connected directly to the control unit, any one type of the units selected from the other two types of the units can be connected directly to the one type of the units and can be connected indirectly to the control unit through the one type of the units, and the other type of the units can be connected directly to the one type of the units selected from the other two types of the units and can be connected indirectly to the control unit through the one type of the units selected from the other two types of the units and also through the one type of the units selected from the three types of the units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above fifteenth aspect, since only one type of the units selected from among the transmitter-receiver units, the display units and the operation units are connected directly to the control unit, a small number of communication lines suffice between the ultrasonographic rooms and the control room.

In a sixteenth aspect of the invention there is provided, in combination with the above fifteenth aspect, an ultrasonic diagnostic apparatus wherein the transmitter-receiver units can be connected directly to the control unit, the display units can be connected directly to the transmitter-receiver units and can be connected indirectly to the control unit through the transmitter-receiver units, and the operation units can be connected directly to the display units and can be connected indirectly to the control unit through the display units and the transmitter-receiver units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above sixteenth aspect, since only the transmitter-receiver units are connected directly to the control unit, a small number of communication lines suffice between the ultrasonographic rooms and the control room.

In a seventeenth aspect of the invention there is provided, in combination with the above sixteenth aspect, an ultrasonic diagnostic apparatus wherein at least one of the operation units and at least one of the display units can be connected directly to the control unit.

According to the ultrasonic diagnostic apparatus in the above seventeenth aspect it is possible to adopt, for example, a mode of use such that a sonographer positioned near a patient uses a display unit connected to a transmitter-receiver unit and an operation unit connected to a display unit and a doctor positioned somewhat away from the patent uses a display unit connected directly to the control unit. This is useful for the doctor to train the sonographer practically.

In an eighteenth aspect of the invention there is provided, in combination with above fifteenth aspect, an ultrasonic diagnostic apparatus wherein the transmitter-receiver units can be connected directly to the control unit, the operation units can be connected directly to the transmitter-receiver units and can be connected indirectly to the control unit through the transmitter-receiver units, and the display units can be connected directly to the operation units and can be connected indirectly to the control unit through the operation units and the transmitter-receiver units.

For example, when one transmitter-receiver unit, one display unit and one operation unit are installed in each of plural ultrasonographic rooms and the control unit is installed in a control room, communication lines are needed between the ultrasonographic rooms and the control room. However, according to the ultrasonic diagnostic apparatus in the above eighteen aspect, since the transmitter-receiver units alone are connected directly to the control unit, a small number of communication lines suffice between the ultrasonographic rooms and the control room.

In a nineteenth aspect of the invention there is provided, in combination with the above eighteenth aspect, an ultrasonic diagnostic apparatus wherein at least one of the operation units and at least one of the display units can be connected directly to the control unit.

According to the ultrasonic diagnostic apparatus in the above nineteenth aspect it is possible to adopt, for example, a mode of use such that a sonographer positioned near a patient uses a display unit connected to an operation unit and an operation unit connected to a transmitter-receiver unit and a doctor positioned somewhat away from the patient uses a display unit and an operation unit both connected directly to the control unit. This is useful for the doctor to train the sonographer practically.

According to the ultrasonic diagnostic apparatus of the invention, since plural transmitter-receiver units, plural display units and plural operation units are connected to one control unit, it is possible to share information and resources. Besides, since the transmitter-receiver units, the display units and the operation units are independent and can be combined freely, the degree of freedom of configuration in actual operation becomes high.

The ultrasonic diagnostic apparatus of the invention is applicable to the case where ultrasonography is performed in plural ultrasonographic rooms simultaneously.

Further objects and advantages of the present invention will be apparent from the following description of the preferred embodiments of the invention as illustrated in the accompanying drawings.

### BRIEF DESCRIPTION OF THE DRAWINGS

FIG. 1 illustrates the configuration of an ultrasonic diagnostic apparatus according to a first embodiment of the invention.

FIG. 2 is a plan view showing the layout of constituent units of the ultrasonic diagnostic apparatus of the first embodiment.

FIG. 3 illustrates the configuration of an ultrasonic diagnostic apparatus according to a second embodiment of the invention.

FIG. 4 is a plan view showing the layout of constituent units of the ultrasonic diagnostic apparatus of the second embodiment.

FIG. 5 illustrates the configuration of an ultrasonic diagnostic apparatus according to a third embodiment of the invention.

FIG. 6 illustrates the configuration of an ultrasonic diagnostic apparatus according to a fourth embodiment of the invention.

FIG. 7 illustrates the configuration of an ultrasonic diagnostic apparatus according to a fifth embodiment of the invention.

FIG. 8 is a plan view showing the layout of constituent units of the ultrasonic diagnostic apparatus of the fifth embodiment.

FIG. 9 illustrates the configuration of an ultrasonic diagnostic apparatus according to a sixth embodiment of the invention.

FIG. 10 illustrates the configuration of an ultrasonic diagnostic apparatus according to a seventh embodiment of the invention.

FIG. 11 illustrates the configuration of an ultrasonic diagnostic apparatus according to an eighth embodiment of the invention.

FIG. 12 illustrates the configuration of an ultrasonic diagnostic apparatus according to a ninth embodiment of the invention.

FIG. 13 illustrates the configuration of an ultrasonic diagnostic apparatus according to a tenth embodiment of the invention.

FIG. 14 illustrates the configuration of an ultrasonic diagnostic apparatus according to an eleventh embodiment of the invention.

FIG. 15 is a plan view showing the layout of constituent elements of the ultrasonic diagnostic apparatus of the eleventh embodiment.

FIG. 16 illustrates the configuration of an ultrasonic diagnostic apparatus according to a twelfth embodiment of the invention.

FIG. 17 illustrates the configuration of an ultrasonic diagnostic apparatus according to a thirteenth embodiment of the invention.

FIG. 18 illustrates the configuration of an ultrasonic diagnostic apparatus according to a fourteenth embodiment of the invention.

FIG. 19 illustrates an ultrasonic diagnostic apparatus according to a fifteenth embodiment of the invention.

FIG. 20 illustrates the configuration of an ultrasonic diagnostic apparatus according to a sixteenth embodiment of the invention.

### DETAILED DESCRIPTION OF THE INVENTION

The invention will be described in more detail by way of embodiments thereof illustrated in the drawings. The invention is not limited by the following embodiments.

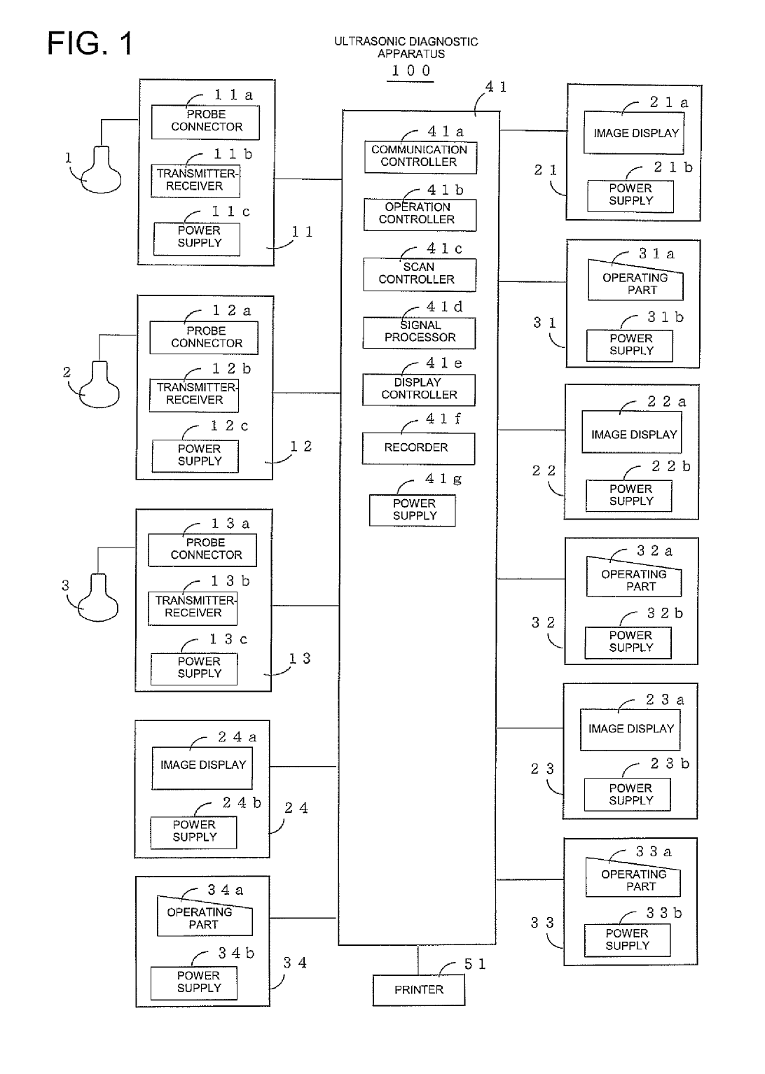

FIG. 1 illustrates the configuration of an ultrasonic diagnostic apparatus 100 according to a first embodiment of the invention.

The ultrasonic diagnostic apparatus 100 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the control unit 41 removably.

The transmitter-receiver units 12 and 13 are also of the same configuration as the transmitter-receiver unit 11. More specifically, transmitter-receiver unit 12 includes a probe connector 12a for connection of the ultrasonic probe 2, a transmitter-receiver 12b adapted to drive the ultrasonic probe 2 and scan the interior of a subject with an ultrasonic beam, and a power supply 12c. The transmitter-receiver unit 12 is connected to the control unit 41 removably. Moreover, transmitter-receiver unit 13 includes a probe connector 13a for connection of the ultrasonic probe 3, a transmitter-receiver 13b adapted to drive the ultrasonic probe 3 and scan the interior of a subject with an ultrasonic beam, and a power supply 13c. The transmitter-receiver unit 13 is connected to the control unit 41 removably.

The display unit 21 includes an image display 21a and a power supply 21b and is connected to the control unit 41 removably.

The display units 22, 23 and 24 are also of the same configuration as the display unit 21. More specifically, display unit 22 includes an image display 22a and a power supply 22b and is connected to the control unit 41 removably. Moreover, display unit 23 includes an image display 23a and a power supply 23b and is connected to the control unit 41 removably. Display unit 24 includes an image display 24a and a power supply 24b and is connected to the control unit 41 removably.

The operation unit 31 includes an operating part 31a, the operating part 31a including a keyboard and a track ball, and a power supply 31b and is connected to the control unit 41 removably.

The operation units 32, 33 and 34 are also of the same configuration as the operation unit 31. More specifically, operation unit 32 includes an operating part 32a, the operating part 32a including a keyboard and a track ball, and a power supply 32b and is connected to the control unit 41 removably. Moreover, operation unit 33 includes an operating part 33a, the operating part 33a including a keyboard and a track ball, and a power supply 33b and is connected to the control unit 41 removably. Further, operation unit 34 includes an operating part 34a, the operating part 34a including a keyboard and a track ball, and a power supply 34b and is connected to the control unit 41 removably.

The control unit 41 includes a communication controller 41a for communication with the transmitter-receiver units 11, 12, 13, the display units 21, 22, 23, 24, the operation units 31, 32, 33, 34 and the printer 51 by wire or wireless, an operation controller 41b which performs an appropriate processing in response to operations of the operation units 31, 32, 33 and 34, a scan controller 41c which scans the interior of the subject with an ultrasonic beam by using the transmitter-receiver units 11, 12, 13 and the ultrasonic probes 1, 2, 3, a signal processor 41d which performs a signal processing such as, for example, generation of an ultrasonic image on the basis of data obtained by the said scanning with an ultrasonic beam, a display controller 41e which displays the ultrasonic image on the corresponding display unit 21, 22, 23, or 24, a recorder 41f which records individual information on the subject, ultrasonic image and imaging parameters and outputs the recorded contents to the printer 51, and a power supply 41g.

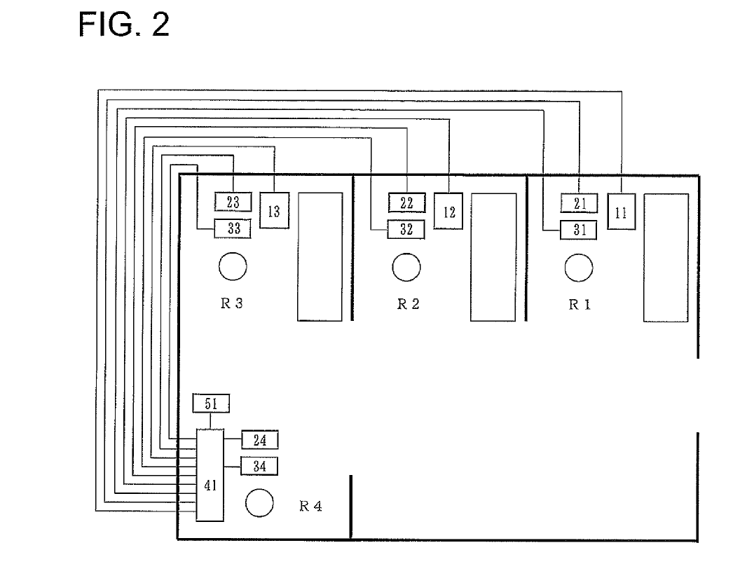

As shown in FIG. 2, in a first ultrasonographic room R1 are disposed the transmitter-receiver unit 11, the display unit 21 and the operation unit 31. In a second ultrasonographic room R2 are disposed the transmitter-receiver unit 12, the display unit 22 and the operation unit 32. In a third ultrasonographic room R3 are disposed the transmitter-receiver unit 13, the display unit 23 and the operation unit 33. In a control room R4 are disposed the display unit 24, the operation unit 34, the control unit 41 and the printer 51. The transmitter-receiver units 11, 12, 13, the display units 21, 22, 23 and the operation units 31, 32, 33 are connected to the control unit 41 through communication lines.

A first sonographer present in the first ultrasonographic room R1 scans a first subject with use of the transmitter-receiver unit 11, the display unit 21 and the operation unit 31. Data obtained by the scanning are transmitted from the transmitter-receiver unit 11 to the control unit 41. On the basis of the transmitted data the control unit 41 generates an ultrasonic image and sends it to the display unit 21. The ultrasonic image thus transmitted is displayed on the display unit 21.

A second sonographer present in the second ultrasonographic room R2 scans a second subject with use of the transmitter-receiver unit 12, the display unit 22 and the operation unit 32. Data obtained by the scanning are transmitted from the transmitter-receiver unit 12 to the control unit 41. On the basis of the transmitted data the control unit 41 generates an ultrasonic image and sends it to the display unit 22. The ultrasonic image thus transmitted is displayed on the display unit 22.

A third sonographer present in the third ultrasonographic room scans a third subject with use of the transmitter-receiver unit 13, the display unit 23 and the operation unit 33. Data obtained by the scanning are transmitted from the transmitter-receiver unit 13 to the control unit 41. On the basis of the transmitted data the control unit 41 generates an ultrasonic image and sends it to the display unit 23. The ultrasonic image thus transmitted is displayed on the display unit 23.

If a doctor present in the ultrasonographic room R4 performs an operation for selecting the first ultrasonographic room R1 with use of the operation unit 34, an image which partially includes the image displayed on the display unit 21 is displayed on the display unit 24. Moreover, it becomes possible to remotely operate the transmitter-receiver unit 11, the display unit 21 and the operation unit 31 from the operation unit 34.

If the doctor present in the control room R4 performs an operation for selecting the second ultrasonographic room R2 with use of the operation unit 34, an image which partially includes the image displayed on the display unit 22 is displayed on the display unit 24. Moreover, it becomes possible to remotely operate the transmitter-receiver unit 12, the display unit 22 and the operation unit 32 from the operation unit 34.

If a doctor present in the control room R4 performs an operation for selecting the third ultrasonographic room R3 with use of the operation unit 34, an image which partially includes the image displayed on the display unit 23 is displayed on the display unit 24. Moreover, it becomes possible to remotely operate the transmitter-receiver unit 13, the display unit 23 and the operation unit 33 from the operation unit 34.

According to the ultrasonic diagnostic apparatus 100 of this first embodiment there are obtained the following effects.

(1) User-preset information is recorded in the control unit 41, so for example when the first sonographer who usually employs the transmitter-receiver unit 11, the display unit 21 and the operation unit 31 disposed in the first ultrasonographic room R1 uses temporarily the transmitter-receiver unit 12, the display unit 22 and the operation unit 32 disposed in the second ultrasonographic room R2, if the user-preset information of the first sonographer recorded in the control unit 41 is read and set into the transmitter-receiver unit 12, the display unit 22 and the operation unit 32, it is possible to proceed with ultrasonography in the same manner as usual.

Only the operation unit 11 is movable, so for example when the first sonographer who usually employs the first ultrasonographic room R1 uses the second ultrasonographic room R2 temporarily, if the operation unit 11 in the first ultrasonographic room R1 is moved to the second ultrasonographic room R2 and the operation unit 12 is replaced by the operation unit 11, it is possible to proceed with ultrasonography with use of the same operation unit 11 as usual even in the second ultrasonographic room R2.

(2) It is less probable that the respective processing loads on the ultrasonographic rooms will become maximum simultaneously in all the ultrasonographic rooms (for example, it is rarely the case that 3D reconstruction or 4D display is performed simultaneously in all the ultrasonographic rooms). Therefore, it is not necessary to let the control unit 41 possess a capability corresponding to the maximum processing load for one ultrasonographic room times the number of ultrasonographic rooms. It suffices for the control unit 41 to have a capability for example about twice as high as the maximum processing load for one ultrasonographic room. In the case where the control unit 41 is given a capability for example about twice as high as the maximum processing load for one ultrasonographic room, there is a surplus capability when the total of the processing loads for all the ultrasonographic rooms is smaller than twice the maximum processing load for one ultrasonographic room, so that it is possible to improve the function and processing load.

For example, it is possible to let the control unit have the function of DICOM server, speed up 3D reconstruction, or update 4D display at high speed.

(3) Since a general-purpose server can be used as the control unit 41, it is possible to provide CPU capability, HD capacity and HDD multiplexing in compliance with customers' requests.

(4) Since private information of the subject is recorded in the control unit 41, for example the first subject can be subjected to ultrasonography easily in any ultrasonographic room or with use of any transmitter-receiver unit.

(5) Since it is possible to share even such a peripheral device as the printer 51, it is possible to reduce not only the initial cost but also the maintenance cost.

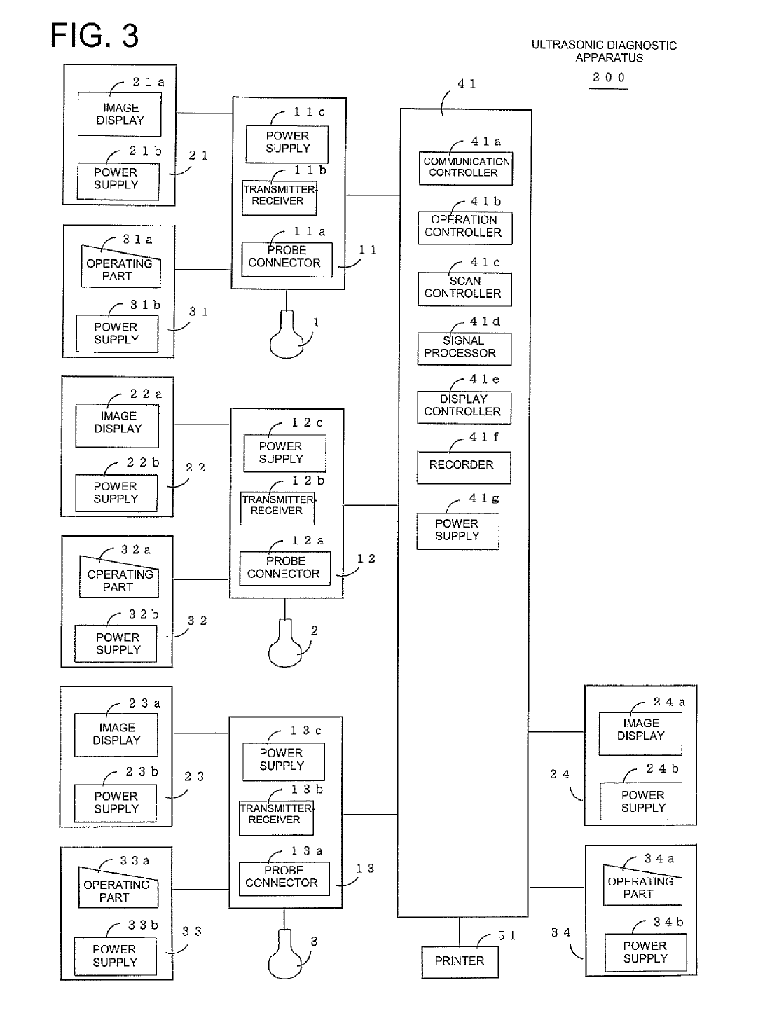

FIG. 3 illustrates the configuration of an ultrasonic diagnostic apparatus 200 according to a second embodiment of the invention.

The ultrasonic diagnostic apparatus 200 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c, and is connected to the control unit 41 removably.

The transmitter-receiver units 12 and 13 are also of the same configuration as the transmitter-receiver unit 11 as described in the first embodiment.

The display unit 21 includes an image display 21a and a power supply 21a and is connected to the transmitter-Receiver unit 11 removably and is thereby connected to the control unit 41 indirectly through the transmitter-receiver unit 11.

As described in the first embodiment, display units 22, 23 and 24 are also of the same configuration as the display unit 21, provided the display unit 24 is connected to the control unit 41 removably.

The operation unit 31 includes an operating part 31a and a power supply 31b and is connected to the transmitter-receiver unit 11 removably and is thereby connected to the control unit 41 indirectly through the transmitter-receiver unit 11.

As described in the first embodiment, operation units 32, 33 and 34 are also of the same configuration as the operation unit 31, provided the operation unit 34 is connected to the control unit 41 removably.

The control unit 41 is of the same configuration as in the first embodiment.

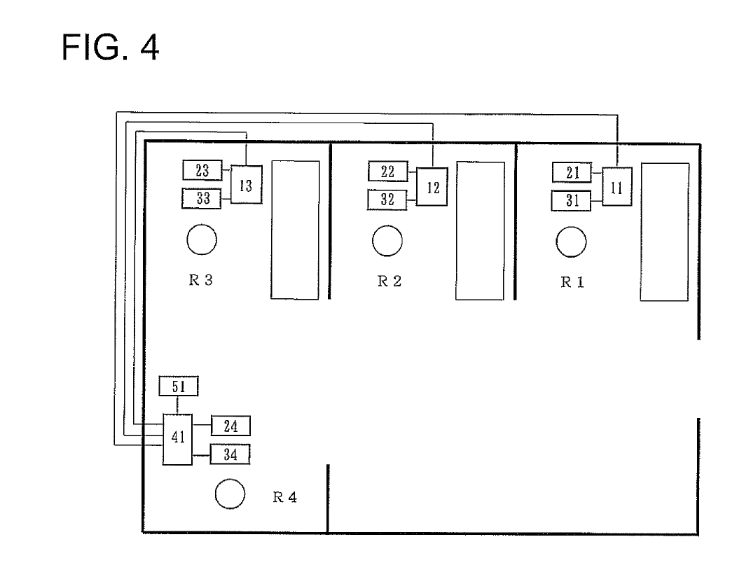

As shown in FIG. 4, in a first ultrasonographic room R1 are disposed the transmitter-receiver unit 11, the display unit 21 and the operation unit 31. In a second ultrasonographic room R2 are disposed the transmitter-receiver unit 12, the display unit 22 and the operation unit 32. In a third ultrasonographic room R3 are disposed the transmitter-receiver unit 13, the display unit 23 and the operation unit 33. In a control room R4 are disposed the display unit 24, the operation unit 34, the control unit 41 and the printer 51. The transmitter-receiver units 11, 12 and 13 are connected to the control unit 41 through communication lines.

A first sonographer present in the first ultrasonographic room R1 scans a first subject with use the transmitter-receiver unit 11, the display unit 21 and the operation unit 31 Data obtained by the scanning are transmitted from the transmitter-receiver unit 11 to the control unit 41. On the basis of the transmitted data the control unit 41 generates an ultrasonic image and sends it to the display unit 21 through the transmitter-receiver unit 11. The ultrasonic image thus transmitted is displayed on the display unit 21.

A second sonographer present in the second ultrasonographic room R2 scans a second subject with use of the transmitter-receiver unit 12, the display unit 22 and the operation unit 32. Data obtained by the scanning are transmitted from the transmitter-receiver unit 12 to the control unit 41. On the basis of the transmitted data the control unit 41 generates an ultrasonic image and sends it to the display unit through the transmitter-receiver unit 12. The ultrasonic image thus transmitted is displayed on the display unit 22.

A third sonographer present in the third ultrasonographic room R3 scans a third subject with use the transmitter-receiver 13, the display unit 23 and the operation unit 33. Data obtained by the scanning are transmitted from the transmitter-receiver unit 13 to the control unit 41. On the basis of the transmitted data the control unit 41 generates an ultrasonic image and sends it to the display unit 23 through the transmitter-receiver unit 13. The ultrasonic image thus transmitted is displayed on the display unit 23.

If a doctor present in the control room R4 performs an operation for selecting the first ultrasonographic room R1 with use of the operation unit 34, an image which partially includes the image displayed on the display unit 21 is displayed on the display unit 24. Moreover, it becomes possible to remotely operate the transmitter-receiver unit 11, the display unit 21 and the operation unit 31 from the operation unit 34.

If the doctor present in the control room R4 performs an operation for selecting the second ultrasonographic room R2 with use of the operation unit 34, an image which partially includes the image displayed on the display unit 22 is displayed on the display unit 24. Moreover, it becomes possible to remotely operate the transmitter-receiver unit 12, the display unit 22 and the operation unit 23 from the operation unit 34.

If the doctor present in the control room R4 performs an operation for selecting the third ultrasonographic room R3 with use of the operation unit 34, an image which partially includes the image displayed on the display unit 23 is displayed on the display unit 24. Moreover, it becomes possible to remotely operate the transmitter-receiver unit 13, the display unit 23 and the operation unit 33 from the operation unit 34.

Also according to the ultrasonic diagnostic apparatus 200 of this second embodiment there are obtained the same effects as in the first embodiment. Besides, a reduced number of communication lines suffice between the ultrasonographic rooms R1, R2, R3 and the control room R4.

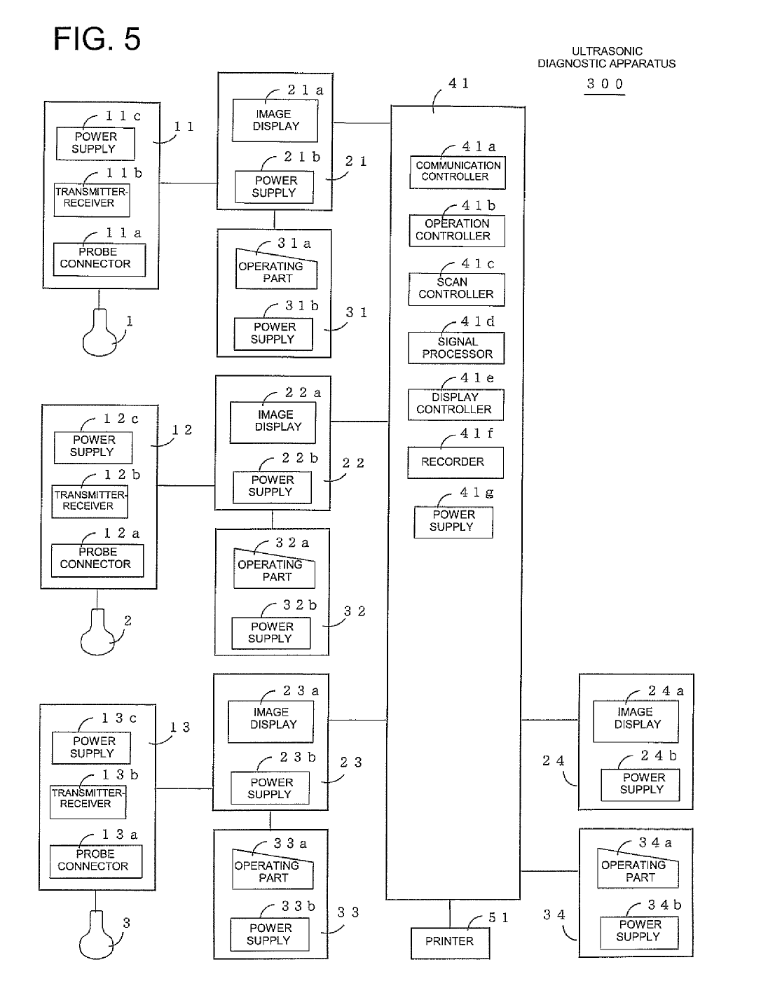

FIG. 5 illustrates the configuration of an ultrasonic diagnostic apparatus according to a third embodiment of the invention.

The ultrasonic diagnostic apparatus 300 includes plural ultrasonic probes 1, 2 and 3, plural transmitter-receiver units 11, 12 and 13 to which the ultrasonic probes 1, 2 and 3 are removably connected respectively, plural display units 21, 22, 23, and 24, plural operation units 31, 32, 33 and 34, one control unit 41, and one printer 51 connected to the control unit 41 removably.

The transmitter-receiver unit 11 includes a probe connector 11a or connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the display unit 21 removably and is thereby connected indirectly to the control unit 41 through the display unit 21.

As described in the first embodiment, transmitter-receiver units 12 and 13 are also of the same configuration as the transmitter-receiver unit 11.

The display unit 21 includes an image display 21a and a power supply 21b and is connected to the control unit 41 removably.

As described in the first embodiment, display units 22, 23 and 24 are also of the same configuration as the display unit 21.

The operation unit 31 includes an operating part 31a and a power supply 31b and is connected to the display unit 21 removably and is thereby connected indirectly to the control unit 41 through the display unit 21.

As described in the first embodiment, operation units 32, 33 and 34 are also of the same configuration as the operation unit 31, provided the operation unit 34 is connected directly to the control unit 41.

The control unit 41 is of the same configuration as in the first embodiment.

The layout of the these units is the same as in FIG. 4, provided the display units 21, 22 and 23 are connected to the control unit 41 through communication lines instead of the transmitter-receiver units 11, 12 and 13 being connected to the control unit 41 through communication lines.

Also according to the ultrasonic diagnostic apparatus 300 of this third embodiment there are obtained the same effects as in the second embodiment.

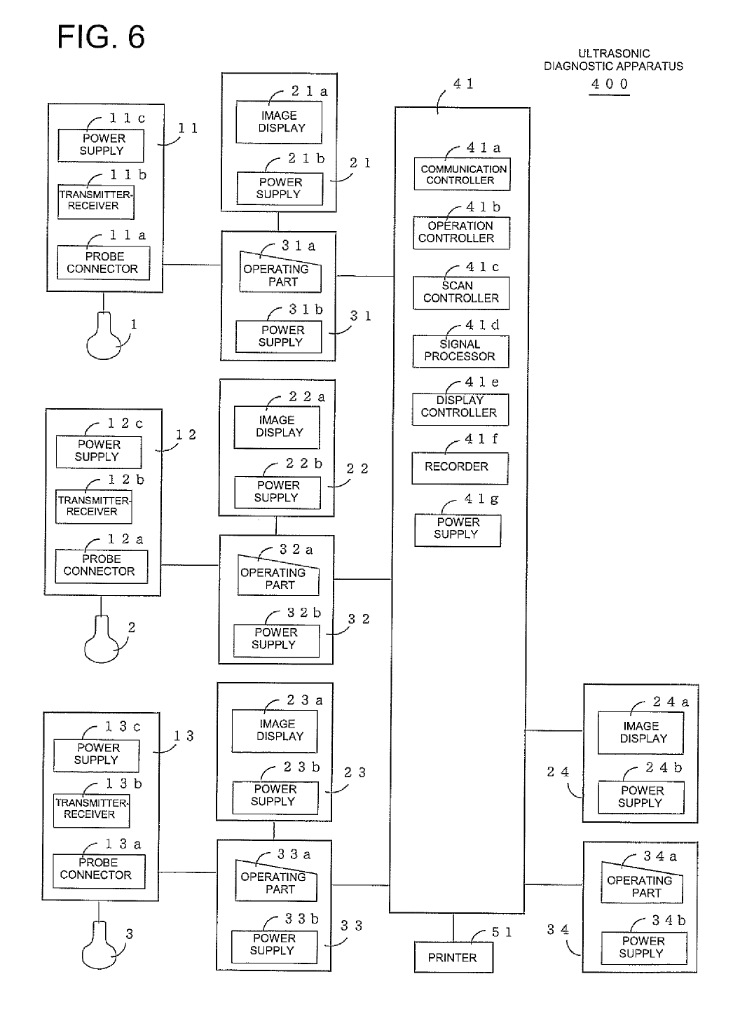

FIG. 6 illustrates the configuration of an ultrasonic diagnostic apparatus 400 according to a fourth embodiment of the invention.

The ultrasonic diagnostic apparatus 400 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is removably connected to the operation unit 31 and is thereby connected indirectly to the control unit 41 through the operation unit 31.

As described in the first embodiment, transmitter-receiver units 12 and 13 are also of the same configuration as the transmitter-receiver unit 11.

The display unit 21 includes an image display 21a and a power supply 21b and is connected to the operation unit 31 removably and is thereby connected indirectly to the control unit 1 through the operation unit 31.

As described in the first embodiment, display units 22, 23 and 24 are also of the same configuration as the display unit 21, provided the display unit 24 is directly connected to the control unit 41.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

The layout of these units is the same as in FIG. 4, provided the operation units 31, 32 and 33 are connected to the control unit 41 through communication lines instead of the transmitter-receiver units 11, 12 and 13 being connected to the control unit 41 through communication lines.

Also according to the ultrasonic diagnostic apparatus 400 of this fourth embodiment there are obtained the same effects as in the second embodiment.

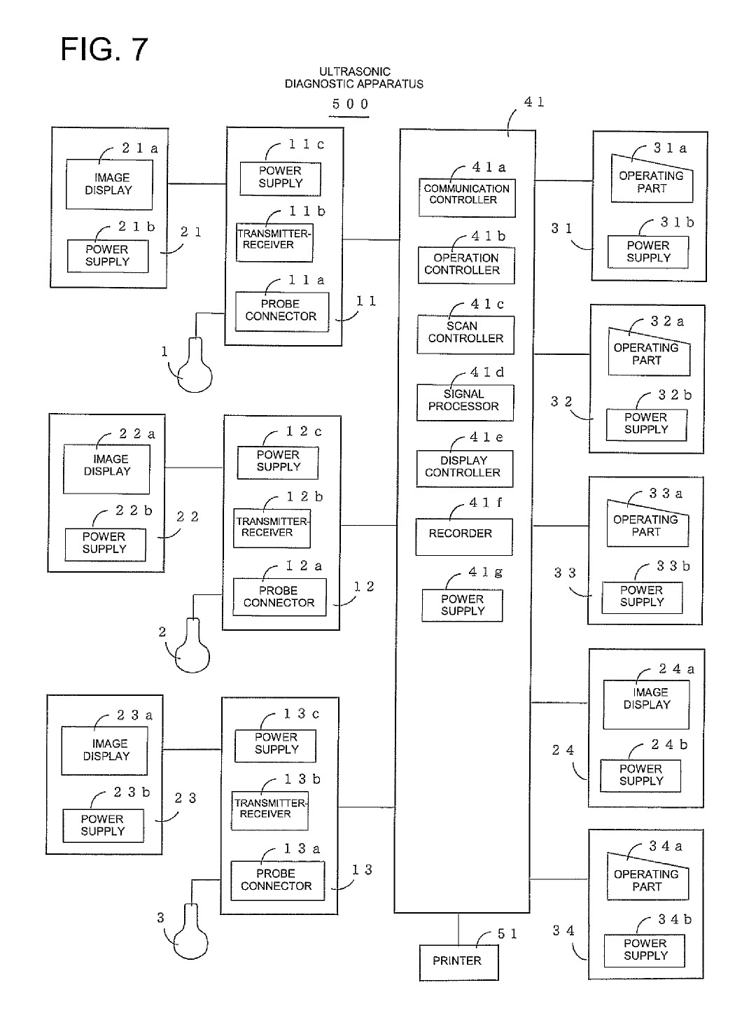

FIG. 7 illustrates the configuration of an ultrasonic diagnostic apparatus 500 according to a fifth embodiment of the invention.

The ultrasonic diagnostic apparatus 500 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the control unit 41 removably.

As described in the first embodiment, transmitter-receiver units 12 and 13 are also of the same configuration as the transmitter-receiver unit 11.

The display unit 21 includes an image display 21a and a power supply 21b and is connected to the transmitter-receiver unit 11 removably and is thereby connected indirectly to the control unit 41 through the transmitter-receiver unit 11.

As described in the first embodiment, display units 22, 23 and 24 are also of the same configuration as the display unit 21, provided the display unit 24 is connected to the control unit 41 removably.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

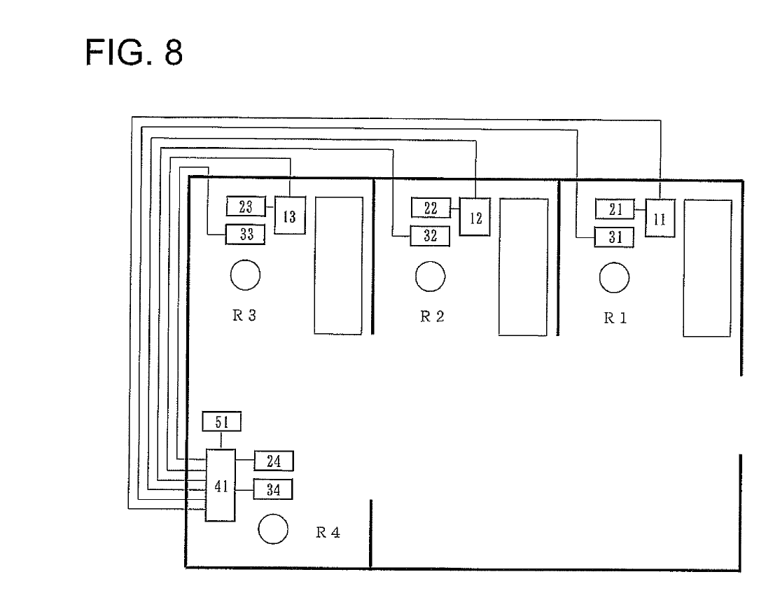

As shown in FIG. 8, in a first ultrasonographic room R1 are disposed the transmitter-receiver unit 11, the display unit 21 and the operation unit 31. In a second ultrasonographic room R2 are disposed the transmitter-receiver unit 12, the display unit 22 and the operation unit 32. In a third ultrasonographic room R3 are disposed the transmitter-receiver unit 13, the display unit 23 and the operation unit 33. In a control room R4 are disposed the display unit 24, the operation unit 34, the control unit 41 and the printer 51. The transmitter-receiver units 11, 12, 13 and the operation units 31, 32, 33 are connected to the control unit 41 through communication lines.

A first sonographer present in the first ultrasonographic room R1 scans a first subject with use of the transmitter-receiver unit 11, the display unit 21 and the operation unit 31. Data obtained by the scanning are transmitted from the transmitter-receiver 11 to the control unit 41. On the basis of the transmitted data the control unit 41 generates an ultrasonic image and sends it to the display unit 21 through the transmitter-receiver unit 11. The ultrasonic image thus transmitted is displayed on the display unit 21.

A second sonographer present in the second ultrasonographic room R2 scans a second subject with use of the transmitter-receiver unit 12, the display unit 22 and the operation unit 32. Data obtained by the scanning are transmitted from the transmitter-receiver 12 to the control unit 41. On the basis of the data thus transmitted the control unit 41 generates an ultrasonic image and sends it to the display unit 22 through the transmitter-receiver unit 12. The ultrasonic image thus transmitted is displayed on the display unit 22.

A third sonographer present in the third ultrasonographic room R3 scans a third subject with use of the transmitter-receiver unit 13, the display unit 23 and the operation unit 33. Data obtained by the scanning are transmitted from the transmitter-receiver unit 11 to the control unit 41. On the basis of the data thus transmitted the control unit 41 generates an ultrasonic image and sends it to the display unit 23 through the transmitter-receiver unit 13. The ultrasonic image thus transmitted is displayed on the display unit 23.

If a doctor present in the control room R4 performs an operation for selecting the first ultrasonographic room R1 with use of the operation unit 34, an image which partially includes the image displayed on the display unit 21 is displayed on the display unit 24. Moreover, it becomes possible to remotely operate the transmitter-receiver unit 11, the display unit 21 and the operation unit 31 through the operation unit 34.

If the doctor present in the control room R4 performs an operation for selecting the second ultrasonogrphic room R2 with use of the operation unit 34, an image which partially includes the image displayed on the display unit 22 is displayed on the display unit 24. Moreover, it becomes possible to remotely operate the transmitter-receiver unit 12, the display unit 22 and the operation unit 32 from the operation unit 34.

If the doctor present in the control room R4 performs an operation for selecting the third ultrasonogrphic room R3 with use of the operation unit 34, an image which partially includes the image displayed on the display unit 23 is displayed on the display unit 24. Moreover, it becomes possible to remotely operate the transmitter-receiver unit 13, the display unit 23 and the operation unit 33 from the operation unit 34.

Also according to the ultrasonic diagnostic apparatus 500 of this fifth embodiment there are obtained the same effects as in the first embodiment. Besides, a small number of communication lines suffice between the ultrasonographic rooms R1, R2, R3 and the control room R4.

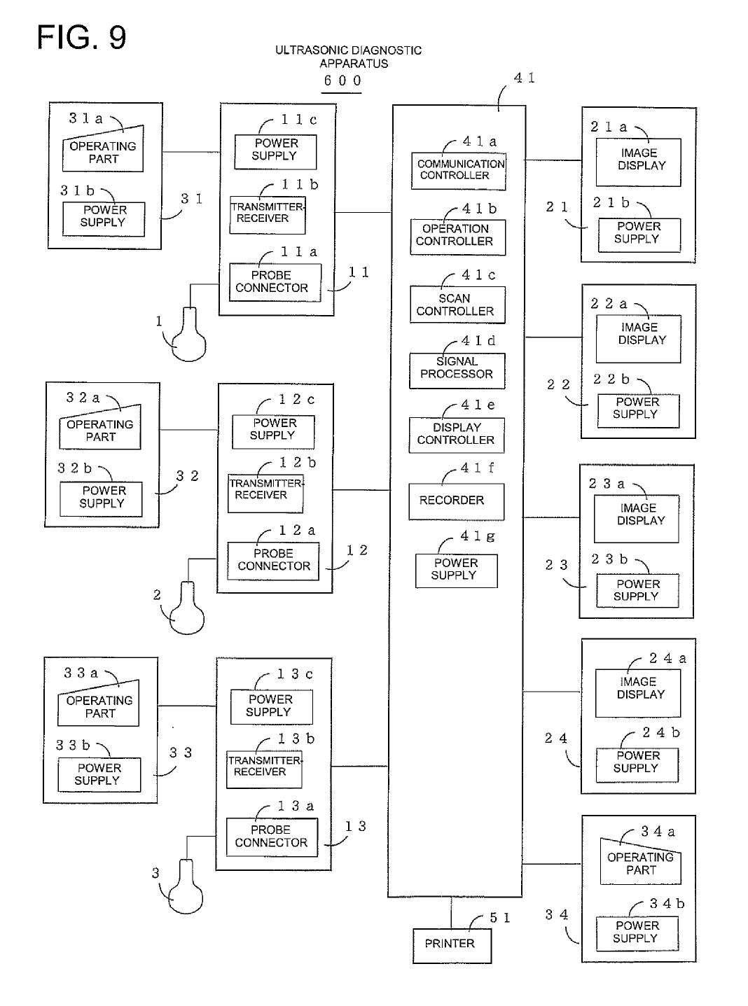

FIG. 9 illustrates the configuration of an ultrasonic diagnostic apparatus 600 according to a sixth embodiment of the invention.

The ultrasonic diagnostic apparatus 600 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the control unit 41 removably.

As described in the first embodiment, transmitter-receiver units 12 and 13 are also of the same configuration as the transmitter-receiver unit 11.

The display unit 21 includes an image display section 21a and a power supply 21b and is connected to the control unit 41 removably.

As described in the first embodiment, display units 22, 23 and 24 are also of the same configuration as the display unit 21.

The operation unit 31 includes an operating part 31a and a power supply 31b and is connected to the transmitter-receiver unit 11 removably and is thereby connected indirectly to the control unit 41 through the transmitter-receiver unit 11.

As described in the first embodiment, operation units 32, 33 and 34 are also of the same configuration as the operation unit 31, provided the operation unit 34 is connected to the control unit 41 removably.

The control unit 41 is of the same configuration as in the first embodiment.

The layout of these units is the same as in FIG. 8. However, the display units 31, 32 and 33 are connected to the control unit 41 through communication lines instead of the operation units 31, 32 and 33 being connected to the control unit 41 through communication lines, and the operation units 31, 32 and 33 are connected to the transmitter-receiver units 11, 12 and 13.

Also according to the ultrasonic diagnostic apparatus 600 of this sixth embodiment there are obtained the same effects as in the fifth embodiment.

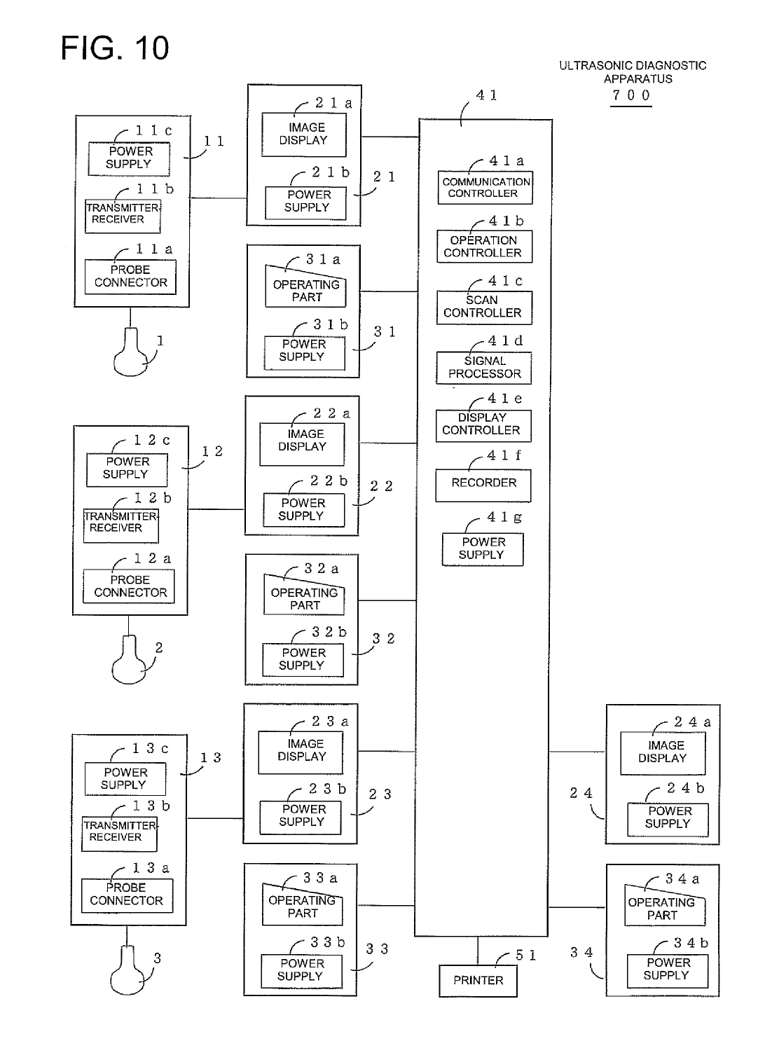

FIG. 10 illustrates the configuration of an ultrasonic diagnostic apparatus 700 according to a seventh embodiment of the invention.

The ultrasonic diagnostic apparatus 700 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the display unit 21 removably and is thereby connected indirectly to the control unit 41 through the display unit 21.

As described in the first embodiment, transmitter-receiver units 12 and 13 are also of the same configuration as the transmitter-receiver unit 11.

The display unit 21 includes an image display 21a and a power supply 21b and is connected to the control unit 41 removably.

As described in the first embodiment, display units 22, 23 and 24 are also of the same configuration as the display unit 21.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

The layout of these units is the same as in FIG. 8, provided the display units 31, 32 and 33 are connected to the control unit 41 through communication lines and the transmitter-receiver units 11, 12 and 13 are connected to the display units 21, 22, and 23, instead of the transmitter-receiver units 11, 12 and 13 being connected to the control unit 41 through communication lines.

Also according to the ultrasonic diagnostic apparatus 700 of this seventh embodiment there are obtained the same effects as in the fifth embodiment.

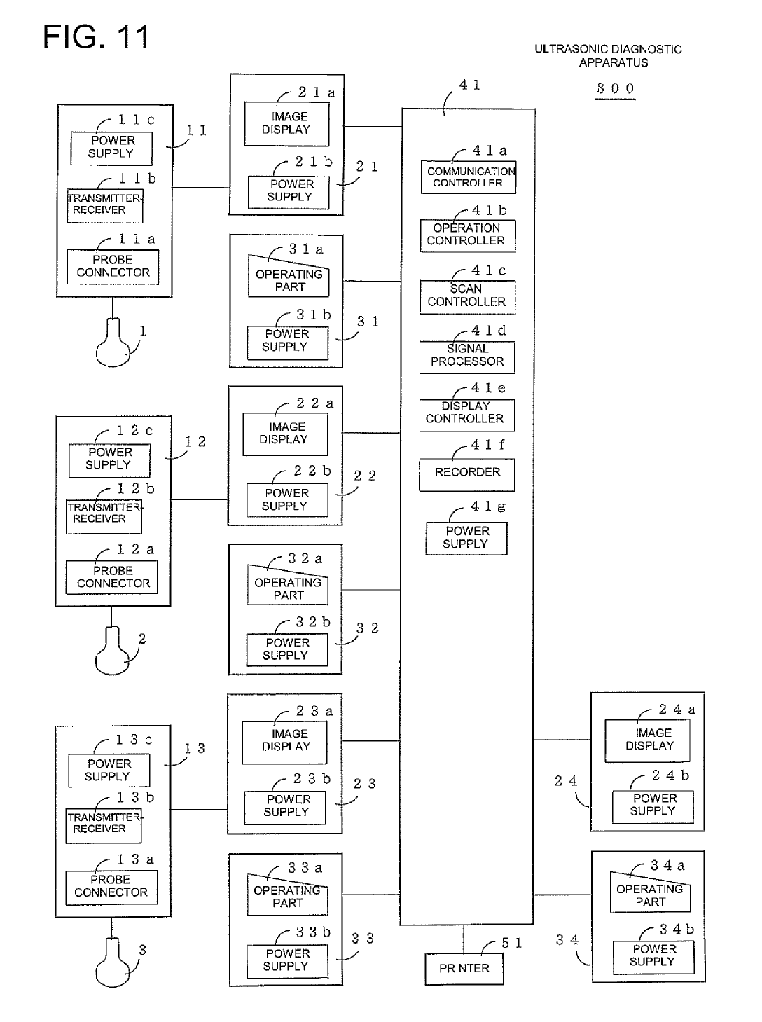

FIG. 11 illustrates the configuration of an ultrasonic diagnostic apparatus 800 according to an eighth embodiment of the invention.

The ultrasonic diagnostic apparatus 800 includes plural ultrasonic probes 1, 2 and 3, plural transmitter-receiver units 11, 12 and 13 connected removably to the ultrasonic probes 1, 2 and 3 respectively, plural display units 21, 22, 23 and 24, plural operation units 31, 32, 33 and 34, one control unit 41, and one printer 51 connected removably to the control unit 41.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the operation unit 31 removably and is thereby connected indirectly to the control unit 41 through the operation unit 31.

As described in the first embodiment, transmitter-receiver units 12 and 13 are also of the same configuration as the transmitter-receiver unit 11.

The display unit 21 includes an image display section 21a and a power supply 21b and is connected to the control unit 41 removably.

As described in the first embodiment, display units 22, 23 and 24 are also of the same configuration as the display unit 21.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

The layout of these units is the same as in FIG. 8, provided the display units 21, 22 and 23 are connected to the control unit 41 through communication lines instead of the transmitter-receiver units 11, 12 and 13 being connected to the control unit 41 through communication lines.

Also according to the ultrasonic diagnostic apparatus 800 of this eighth embodiment there are obtained the same effects as in the fifth embodiment.

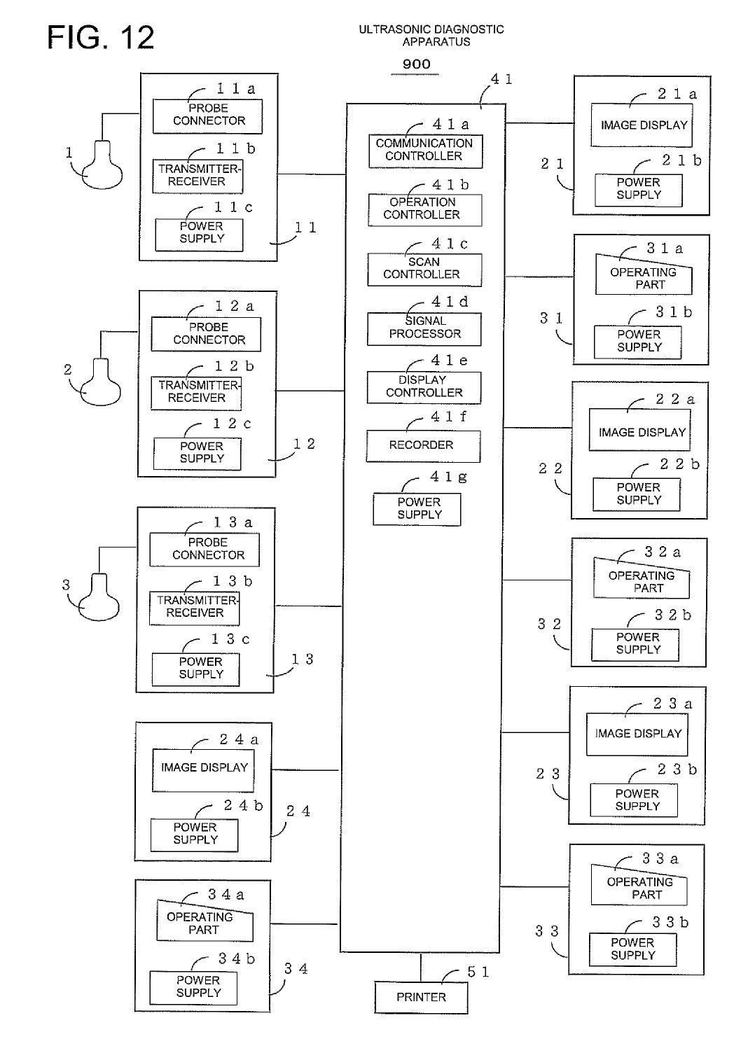

FIG. 12 illustrates the configuration of an ultrasonic diagnostic apparatus 900 according to a ninth embodiment of the invention.

The ultrasonic diagnostic apparatus 900 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with use of an ultrasonic beam, and a power supply 11c. The transmitter-receiver unit 11 is connected to the control unit 41 removably.

As described in the first embodiment, transmitter-receiver units 12 and 13 are also of the same configuration as the transmitter-receiver unit 11.

The display unit 21 includes an image display 21a and a power supply 21b and is connected to the operation unit 31 removably and is thereby connected indirectly to the control unit 41 through the operation unit 31.

As described in the first embodiment, display units 22, 23 and 24 are also of the same configuration as the display unit 21, provided the display unit 24 is connected to the control unit 41 removably.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

The layout of these units is the same as in FIG. 8, provided the display units 21, 22 and 23 are connected to the operation units 31, 32 and 33, respectively, instead of being connected to the transmitter-receiver units 11, 12 and 13.

Also according to the ultrasonic diagnostic apparatus 900 of this ninth embodiment there are obtained the same effects as in the fifth embodiment.

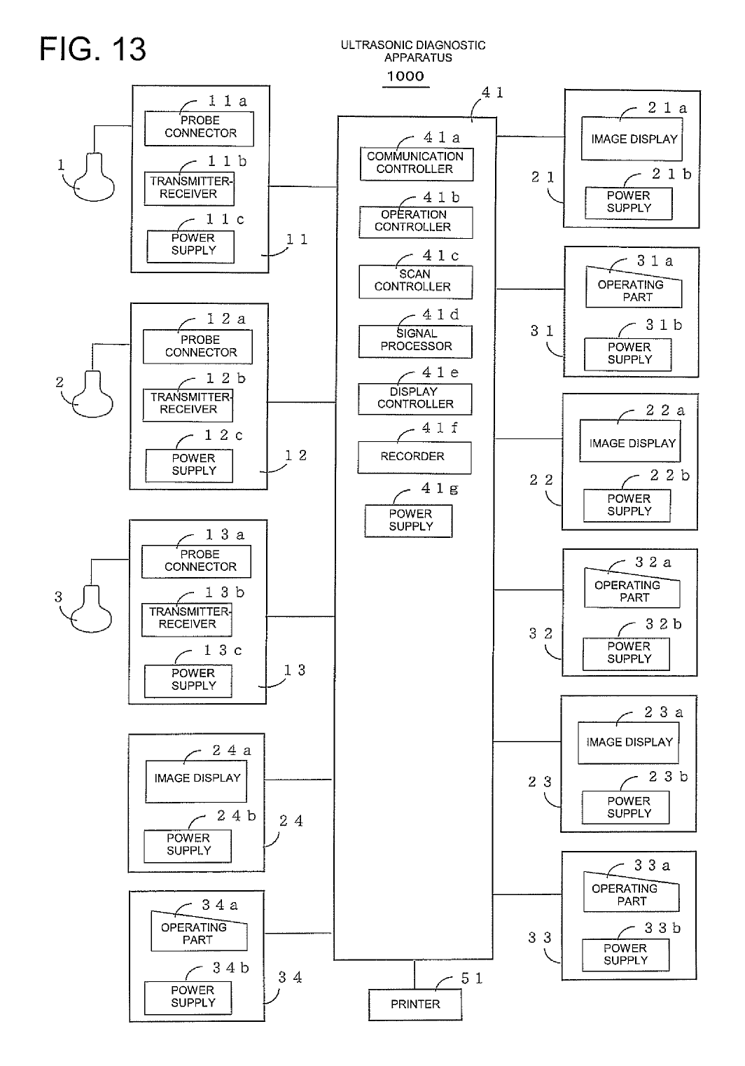

FIG. 13 illustrates the configuration of an ultrasonic diagnostic apparatus 1000 according to a tenth embodiment of the invention.

The ultrasonic diagnostic apparatus 1000 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the control unit 41 removably.

As described in the first embodiment, transmitter-receiver units 12 and 13 are also of the same configuration as the transmitter-receiver unit 11.

The display unit 21 includes an image display 21a and a power supply 21b and is connected to the control unit 41 removably.

As described in the first embodiment, display units 22, 23 and 24 are also of the same configuration as the display unit 21.

The operation unit 31 includes an operating part 31a and a power unit 31b and is connected to the display unit 21 removably and is thereby connected indirectly to the control unit 41 through the display unit 21.

As described in the first embodiment, operation units 32, 33 and 34 are also of the same configuration as the operation unit 31, provided the operation unit 34 is connected to the control unit 41 removably.

The control unit 41 is of the same configuration as in the first embodiment.

The layout of these units is the same as in FIG. 8, provided the operation units 31, 32 and 33 are connected to the display units 21, 22 and 23, respectively, instead of being connected to the control unit 41 and the display units 21, 22 and 23 are connected to the control unit 41 instead of being connected to the transmitter-receiver units 11, 12 and 13.

Also according to the ultrasonic diagnostic apparatus 1000 of this tenth embodiment there are obtained the same effects as in the fifth embodiment.

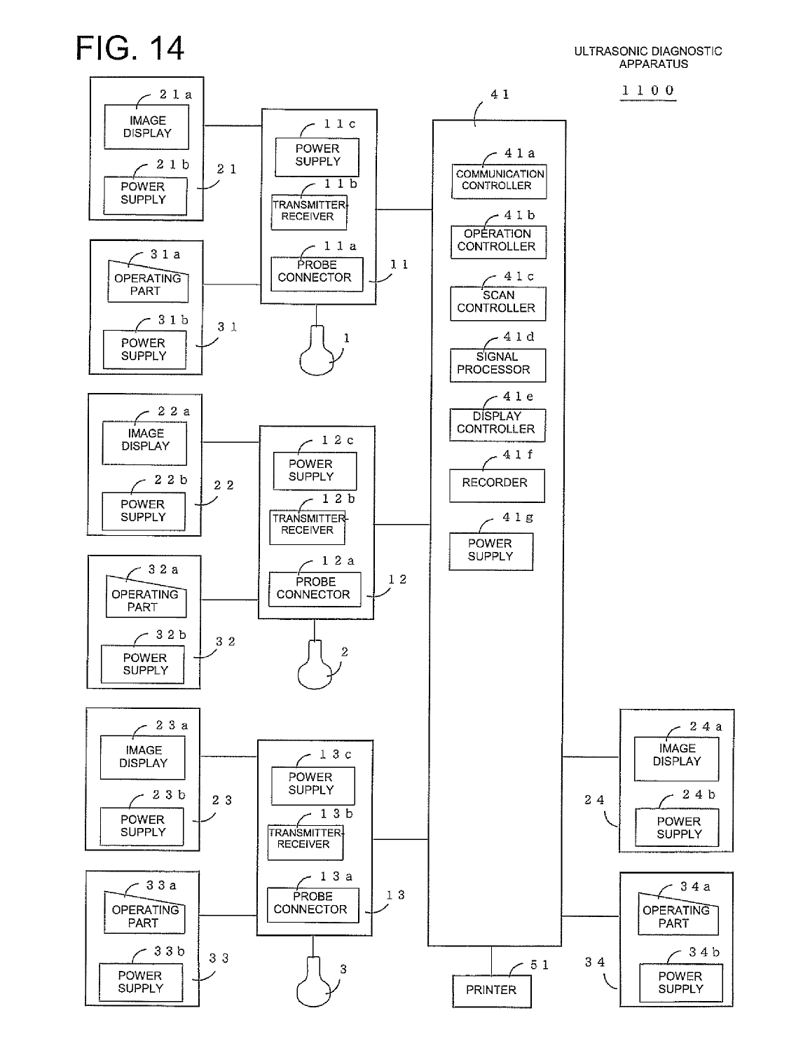

FIG. 14 illustrates the configuration of an ultrasonic diagnostic apparatus 1100 according to an eleventh aspect of the invention.

The ultrasonic diagnostic apparatus 1100 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with use of an ultrasonic beam, and a power supply 11c. The transmitter-receiver unit 11 is connected to the control unit 41 removably.

As described in the first embodiment, transmitter-receiver units 12 and 13 are also of the same configuration as the transmitter-receiver unit 11.

The display unit 21 includes an image display 21a and a power supply 21b and is connected to the transmitter-receiver unit 11 removably and is thereby connected indirectly to the control unit 41 through the transmitter-receiver unit 11.

As described in the first embodiment, display units 22, 23 and 24 are also of the same configuration as the display unit 21, provided the display unit 24 is connected to the control unit 41 removably.

The operation unit 31 includes an operating part 31a and a power unit 31b and is connected to the display unit 21 removably and is thereby connected indirectly to the control unit 41 through the transmitter-receiver unit 11 and the display unit 21.

As described in the first embodiment, operation units 32, 33 and 34 are also of the same configuration as the operation unit 31, provided the operation unit 34 is connected to the control unit 41 removably.

The control unit 41 is of the same configuration as in the first embodiment.

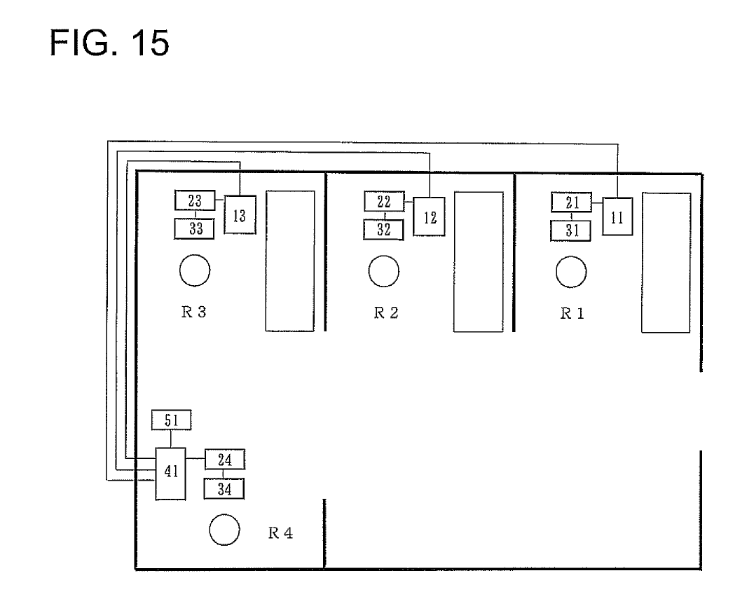

As shown in FIG. 15, in a first ultrasonographic room R1 are disposed the transmitter-receiver unit 11, the display unit 21 and the operation unit 31. In a second ultrasonographic room R2 are disposed the transmitter-receiver unit 12, the display unit 22 and the operation unit 32. In a third ultrasonographic room R3 are disposed the transmitter-receiver unit 13, the display unit 23 and the operation unit 33. In a control room R4 are disposed the display unit 24, the operation unit 34, the control unit 41 and the printer 51. The transmitter-receiver units 11, 12 and 13 are connected to the control unit 41 through communication lines.

A first sonographer present in the first ultrasonographic room R1 scans a first subject with use of the transmitter-receiver unit 11, the display unit 21 and the operation unit 31. Data obtained by the scanning are transmitted from the transmitter-receiver unit 11 to the control unit 41. On the basis of the transmitted data the control unit 41 generates an ultrasonic image and sends it to the display unit 21 through the transmitter-receive unit 11. The ultrasonic image thus transmitted is displayed on the display unit 21.

A second sonographer present in the second ultrasonographic room R2 scans a second subject with use of the transmitter-receiver unit 12, the display unit 22 and the operation unit 32. Data obtained by the scanning are transmitted from the transmitter-receiver unit 12 to the control unit 41. On the basis of the transmitted data the control unit 41 generates an ultrasonic image and sends it to the display unit 22 through the transmitter-receiver unit 12. The ultrasonic image thus transmitted is displayed on the display unit 22.

A third sonographer present in the third ultrasonographic room R3 scans a third subject with use of the transmitter-receiver unit 13, the display unit 23 and the operation unit 33. Data obtained by the scanning are transmitted from the transmitter-receiver unit 13 to the control unit 41. On the basis of the transmitted data the control unit 41 generates an ultrasonic image and sends it to the display unit 23 through the transmitter-receiver unit 13. The ultrasonic image thus transmitted is displayed on the display unit 23.

If a doctor present in the control room R4 performs an operation for selecting the first ultrasonographic room R1 with use of the operation unit 34, an image which partially includes the image displayed on the display unit 21 is displayed on the display unit 24. Moreover, it becomes possible remotely operate the transmitter-receiver unit 11, the display unit 21 and the operation unit 31 from the operation unit 34.

If the doctor present in the control room R4 performs an operation for selecting the second ultrasonographic room R2 with use of the operation unit 34, an image which partially includes the image displayed on the display unit 22 is displayed on the display unit 24. Moreover, it becomes possible to remotely operate the transmitter-receiver unit 12, the display unit 22 and the operation unit 32 from the operation unit 34.

If the doctor present in the control room R4 performs an operation for selecting the third ultrasonographic room R3 with use of the operation unit 34, an image which partially includes the image displayed on the display unit 23 is displayed on the display unit 24. Moreover, it becomes possible to remotely operate the transmitter-receiver unit 13, the display unit 23 and the operation unit 33 from the operation unit 34.

Also according to the ultrasonic diagnostic apparatus 1200 of this twelfth embodiment there are obtained the same effects as in the second embodiment.

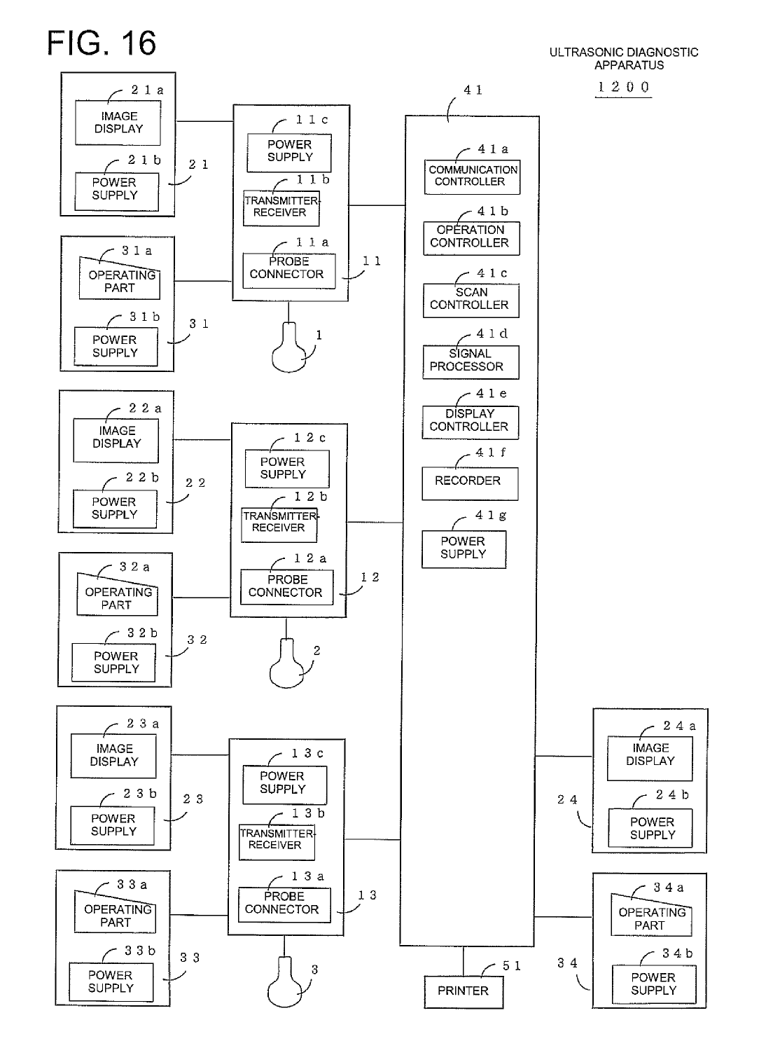

FIG. 16 illustrates the configuration of an ultrasonic diagnostic apparatus 1200 according to a twelfth embodiment of the invention.

The ultrasonic diagnostic apparatus 1200 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the control unit 41 removably.

As described in the first embodiment, transmitter-receiver units 12 and 13 are also of the same configuration as the transmitter-receiver unit 11.

The display unit 21 includes an image display section 21a and a power supply 21b and is connected to the operation unit 31 removably and is thereby connected indirectly to the control unit 41 through the transmitter-receiver unit 11 and the operation unit 31.

As described in the first embodiment, display units 22, 23 and 24 are also of the same configuration as the display unit 21, provided the display unit 24 is connected to the control unit 41 removably.

The operation unit 31 includes an operating part 31a and a power supply 31b and is connected to the transmitter-receiver unit 11 removably and is thereby connected indirectly to the control unit 41 through the transmitter-receiver unit 11.

As described in the first embodiment, operation units 32, 33 and 34 are also of the same configuration as the operation unit 31, provided the operation unit 34 is connected to the control unit 41 removably.

The control unit 41 is of the same configuration as in the first embodiment.

The layout of these units is the same as in FIG. 15, provided the operation units 31, 32 and 33 are connected to the transmitter-receiver units 11, 12 and 13 instead of the display units 21, 22 and 23 being connected to the control unit 41.

Also according to the ultrasonic diagnostic apparatus 1200 of this twelfth embodiment there are obtained the same effects as in the eleventh embodiment.

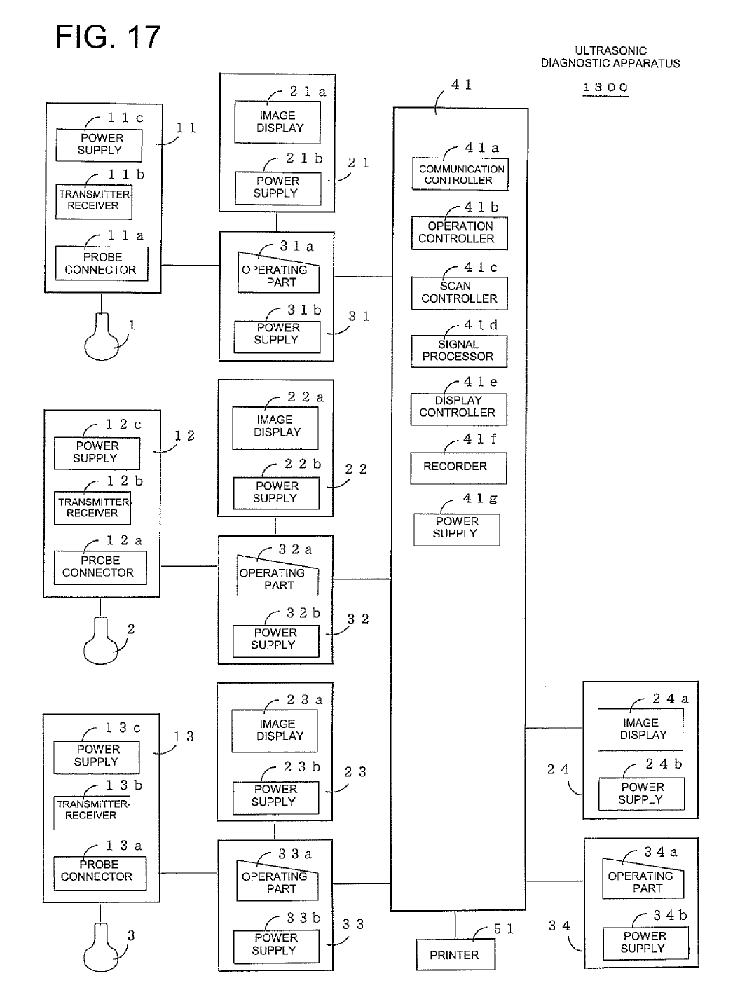

FIG. 17 illustrates the configuration of an ultrasonic diagnostic apparatus 1300 according to a thirteenth embodiment of the invention.

The ultrasonic diagnostic apparatus 1300 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receive unit 11 is connected to the operation unit 31 and is thereby connected indirectly to the control unit 41 through the display unit 21 and the operation unit 31.

As described in the first embodiment, transmitter-receiver units 12 and 13 are also of the same configuration as the transmitter-receiver unit 11.

The display unit 21 includes an image display 31a and a power supply 21b and is connected to the control unit 41 removably.

As described in the first embodiment, display units 22, 23 and 24 are also of the same configuration as the display unit 21.

The operation unit 31 includes an operating part 31a and a power supply 31b and is connected to the display unit 21 removably and is thereby connected indirectly to the control unit 41 through the display unit 21.

As described in the first embodiment, operation units 32, 33 and 34 are also of the same configuration as the operation unit 31, provided the operation unit 34 is connected to the control unit 41 removably.

The control unit 41 is of the same configuration as in the first embodiment.

The layout of these units is the same as in FIG. 15, provided the transmitter-receiver units 11, 12 and 13 are connected to the operation units 31, 32 and 33 instead of being connected to the control unit 41 and the display units 21, 22 and 23 are connected to the control unit 41 instead of being connected to the transmitter-receiver units 11, 12 and 13.

Also according to the ultrasonic diagnostic apparatus 1300 of this thirteenth embodiment there are obtained the same effects as in the eleventh embodiment.

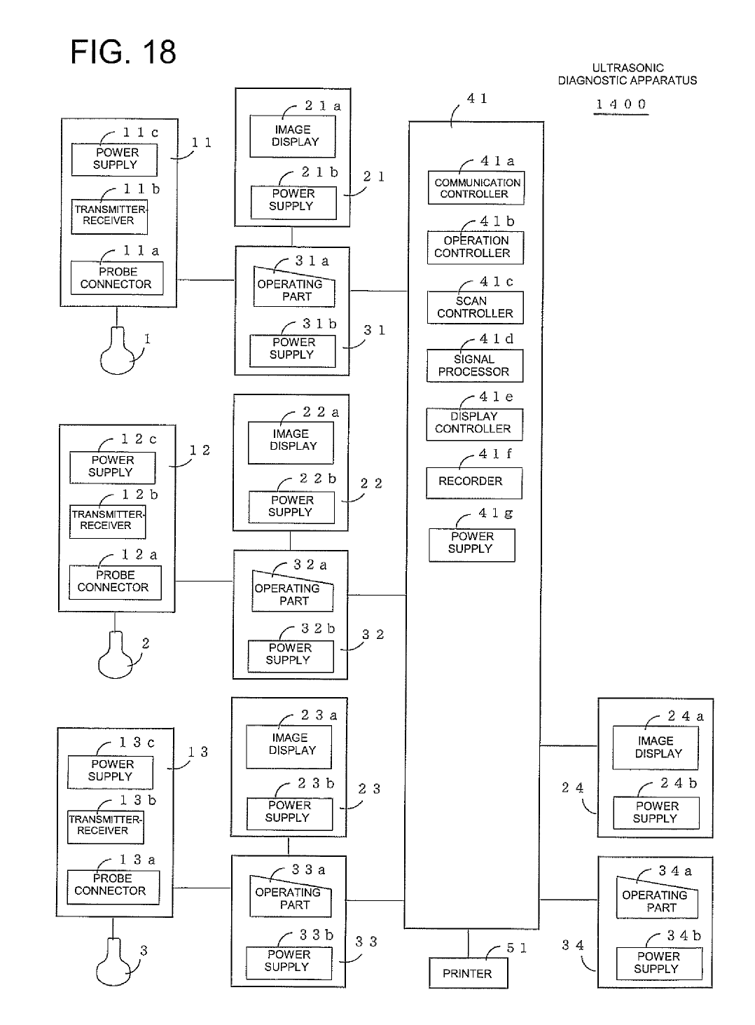

FIG. 18 illustrates the configuration of an ultrasonic diagnostic apparatus 1400 according to a fourteenth embodiment of the invention.

The ultrasonic diagnostic apparatus 1400 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and once printer 51 connected to the control unit 41 removably.

The transmitter-receiver unit 11 includes a probe connector 11a for connection of the ultrasonic probe 1, a transmitter-receiver section 11b adapted to drive the ultrasonic probe 1 and scan the interior of a subject with an ultrasonic beam, and a power supply 11c. The transmitter-receiver unit 11 is connected to the display unit 21 removably and is thereby connected indirectly to the control unit 41 through the display unit 21.

As described in the first embodiment, transmitter-receiver units 12 and 13 are also of the same configuration as the transmitter-receiver unit 11.

The display unit 21 includes an image display 21a and a power supply 21b and is connected to the control unit 41 removably.

As described in the first embodiment, display units 22, 23 and 24 are also of the same configuration as the display unit 21.

The operation unit 31 includes an operating part 31a and a power supply 31b and is connected to the transmitter-receiver unit 11 removably and is thereby connected indirectly to the control unit 41 through the transmitter-receiver unit 11 and the display unit 21.

As described in the first embodiment, operation units 32, 33 and 34 are also of the same configuration as the control unit 31, provided the operation unit 34 is connected to the control unit 41 removably.

The control unit 41 is of the same configuration as in the first embodiment.

The layout of these units is the same as in FIG. 15, provided the display units 21, 22 and 23 are connected to the control unit 41 instead of the transmitter-receiver units 11, 12 and 13 being connected to the control unit 41 and the operation units 31, 32 and 33 are connected to the transmitter-receiver units 11, 12 and 13 instead of being connected to the display units 21, 22 and 23.

Also according to the ultrasonic diagnostic apparatus 1400 of this fourteenth embodiment there are obtained the same effects as in the eleventh embodiment.

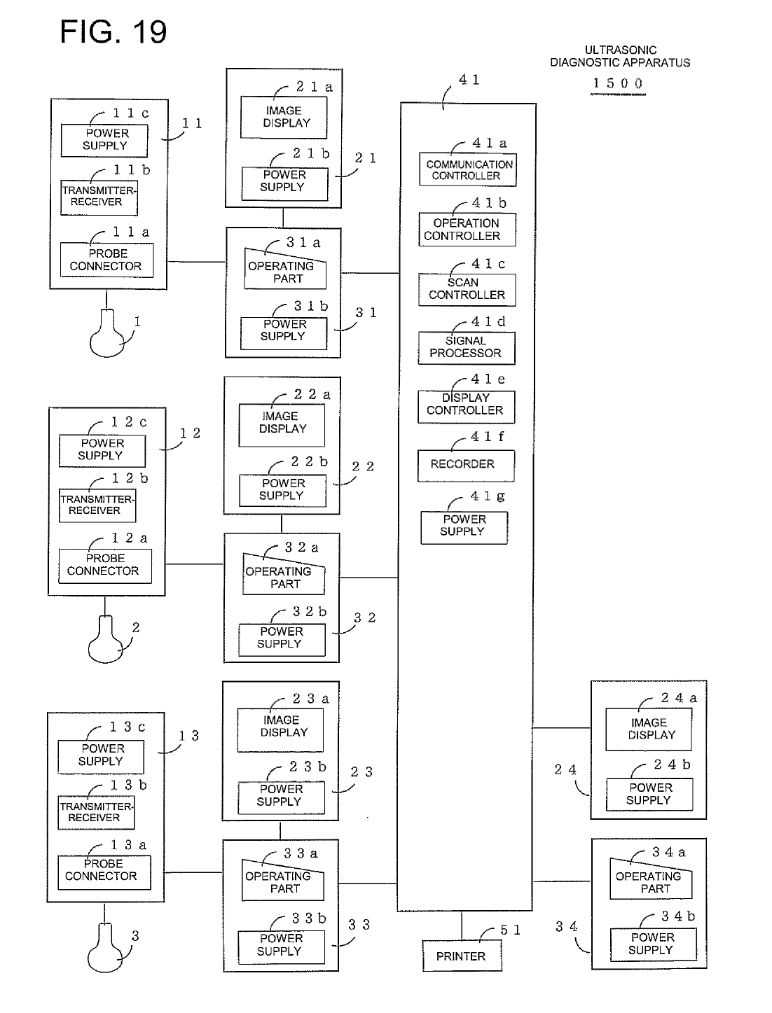

FIG. 19 illustrates the configuration of an ultrasonic diagnostic apparatus 1500 according to a fifteenth embodiment of the invention.

The ultrasonic diagnostic apparatus 1500 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver 11b adapted to drive the ultrasonic probe 1 and scan the interior of a subject with an ultrasonic beam, and a power supply 11c. The transmitter-receiver unit 11 is connected to the display unit 21 removably and is thereby connected indirectly to the control unit 41 through the display unit 21 and the operation unit 31.

As described in the first embodiment, transmitter-receiver units 12 and 13 are also of the same configuration as the transmitter-receiver unit 11.

The display unit 21 includes an image display 21a and a power supply 21b and is connected to the operation unit 31 removably and is thereby connected indirectly to the control unit 41 through the operation unit 31.

As described in the first embodiment, display units 22, 23 and 24 are also of the same configuration as the display unit 21, provided the display unit 24 is connected to the control unit 41 removably.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

The layout of these units is the same as in FIG. 15, provided the operation units 31, 32 an 33 are connected to the control unit 41 instead of the transmitter-receiver units 11, 12 and 13 being connected to the control unit 41.

Also according to the ultrasonic diagnostic apparatus 1500 of this fifteenth embodiment there are obtained the same effects as in the eleventh embodiment.

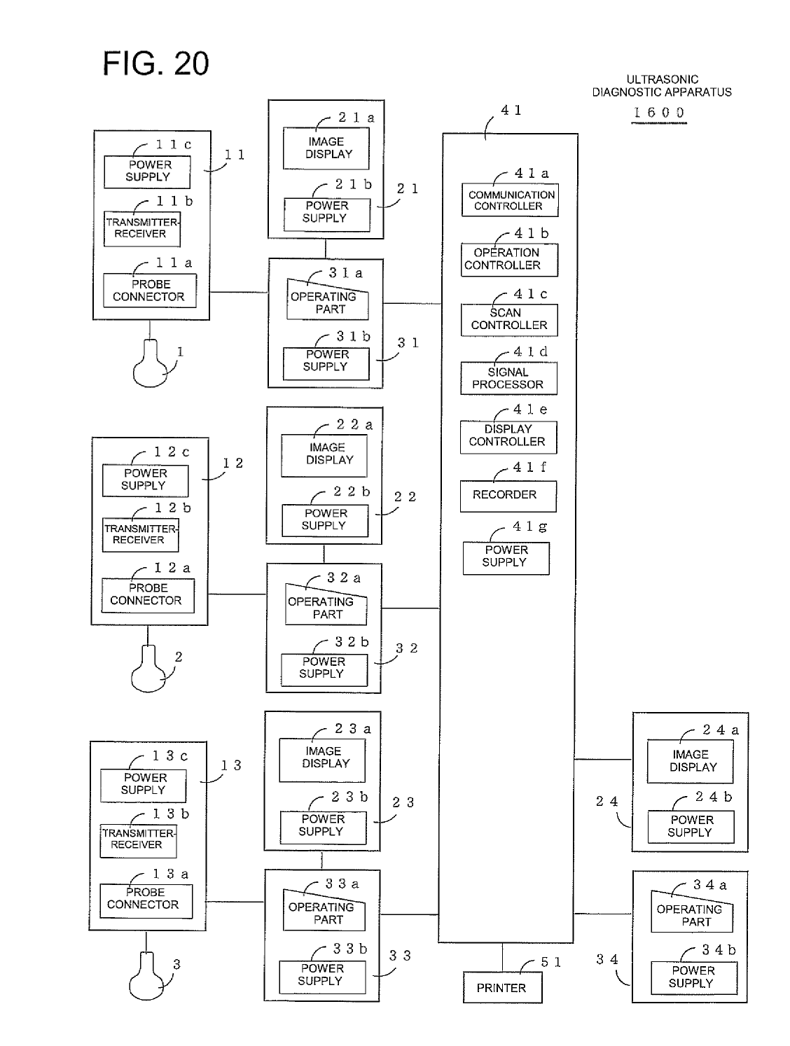

FIG. 20 illustrates the configuration of an ultrasonic diagnostic apparatus 1600 according to a sixteenth embodiment of the invention.

The ultrasonic diagnostic apparatus 1600 includes plural ultrasonic probes 1, 2 and 3, plural transmitter-receiver units 11, 12 and 13 to which the ultrasonic probes 1, 2 and 3 are removably connected respectively, plural display units 21, 22, 23 and 24, plural operation units 31, 32, 33 and 34, one control unit 41, and one printer 51 connected to the control unit 41 removably.

The transmitter-receiver unit 11 includes a probe connector 11a for connection of the ultrasonic probe 1, a transmitter-receiver section 11b adapted to drive the ultrasonic probe and scan the interior of a subject with an ultrasonic beam, and a power supply 11c. The transmitter-receiver unit 11 is connected to the operation unit 31 removably and is thereby connected indirectly to the control unit 41 through the operation unit 31.

As described in the first embodiment, transmitter-receiver units 12 and 13 are also of the same configuration as the transmitter-receiver unit 11.

The display unit 21 includes an image display 21a and a power supply 21b and is connected to the transmitter-receiver unit 11 removably and is thereby connected indirectly to the control unit 41 through the transmitter-receive unit 11 and the operation unit 31.

As described in the first embodiment, display units 22, 23 and 24 are of the same configuration as the display unit 21, provided the display unit 24 is connected to the control unit 41 removably.

The operation unit 31 includes an operating part 31a and a power supply 31b and is connected to the control unit 41 removably.

As described in the first embodiment, operation units 32, 33 and 34 are also of the same configuration as the operation unit 31.

The control unit 41 is of the same configuration as in the first embodiment.

The layout of these units is the same as in FIG. 15, provided the operation units 31, 32 and 33 are connected to the control unit 41 instead of the transmitter-receiver units 11, 12 and 13 being connected to the control unit 41 and the transmitter-receiver units 11, 12 and 13 are connected to the operation units 31, 32 and 33 instead of the display units 21, 22 and 23 being connected to the operation units 31, 32 and 33.

Also according to the ultrasonic diagnostic apparatus 1600 of this sixteenth embodiment there are obtained the same effects as in the eleventh embodiment.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a plurality of transmitter-receiver units, each transmitter-receiver unit comprising a probe connector, a transmitter-receiver and a power supply;
a plurality of display units, each display unit comprising an image display and a power supply;
a plurality of operation units, each operation unit comprising an operating part and a power supply; and
a control unit, wherein said plurality of transmitter-receiver units, said plurality of display units, and said plurality of operation units are connected to said control unit, and said control unit is configured to independently control said plurality of plural transmitter-receiver units, said plurality of plural display units, and said plurality of plural operation units.

2. An ultrasonic diagnostic apparatus according to claim 1, wherein said plurality of transmitter-receiver units, said plurality of display units, and said plurality of operation units are connected directly to said control unit.

3. An ultrasonic diagnostic apparatus according to claim 1, wherein a first type of unit selected from among said plurality of transmitter-receiver units, said plurality of display units, and said plurality of operation units is connected directly to said control unit, and a second type of unit and a third type of unit, each different from the first type of unit, are connected directly to the first type of unit and are connected indirectly to said control unit through the first type of unit.

4. An ultrasonic diagnostic apparatus according to claim 3, wherein said plurality of transmitter-receiver units are connected directly to said control unit, and said plurality of display units and said plurality of operation units are connected directly to said plurality of transmitter-receiver units such that said plurality of display units and said plurality of operations units are connected indirectly to said control unit through said plurality of transmitter-receiver units.

5. An ultrasonic diagnostic apparatus according to claim 4, wherein at least one display unit of said plurality of display units and at least one operation unit of said plurality of operation units are connected directly to said control unit.

6. An ultrasonic diagnostic apparatus according to claim 1, wherein a first type of unit and a second type of unit selected from among said plurality of transmitter-receiver units, said plurality of display units, and said plurality of operation units are connected directly to said control unit, and a third type of unit, different than the first type of unit and the second type of unit, is connected directly to one of the first type of unit and the second type of unit and is thus connected indirectly to said control unit through the one of the first type of unit and the second type of unit.

7. An ultrasonic diagnostic apparatus according to claim 6, wherein said plurality of transmitter-receiver units and said plurality of operation units are connected directly to said control unit, and said plurality of display units are connected directly to said plurality of transmitter-receiver units and are thus connected indirectly to said control unit through said plurality of transmitter-receiver units.

8. An ultrasonic diagnostic apparatus according to claim 7, wherein at least one display unit of said plurality of display units is connected directly to said control unit.

9. An ultrasonic diagnostic apparatus according to claim 6, wherein said plurality of transmitter-receiver units and said plurality of display units are connected directly to said control unit, and said plurality of operation units are connected directly to said plurality of transmitter-receiver units and are thus connected indirectly to said control unit through said plurality of transmitter-receiver units.

10. An ultrasonic diagnostic apparatus according to claim 9, wherein at least one operation unit of said plurality of operation units is connected directly to said control unit.

11. An ultrasonic diagnostic apparatus according to claim 6, wherein said plurality of transmitter-receiver units and said plurality of operation units are connected directly to said control unit, and said plurality of display units are connected directly to said plurality of operation units and are thus connected indirectly to said control unit through said plurality of operation units.

12. An ultrasonic diagnostic apparatus according to claim 11, wherein at least one display unit of said plurality of display units is connected directly to said control unit.

13. An ultrasonic diagnostic apparatus according to claim 6, wherein said plurality of transmitter-receiver units and said plurality of display units are connected directly to said control unit, and said plurality of operation units are connected directly to said plurality of display units and are thus connected indirectly to said control unit through said plurality of display units.

14. An ultrasonic diagnostic apparatus according to claim 13, wherein at least one operation unit of said plurality of operation units is connected directly to said control unit.

15. An ultrasonic diagnostic apparatus according to claim 1, wherein a first type of unit selected from among said plurality of transmitter-receiver units, said plurality of display units, and said plurality of operation units is connected directly to said control unit, a second type of unit, different from the first type of unit, is connected directly to the first type of unit and is thus connected indirectly to said control unit through the first type of unit, and a third type of unit, different from the first type of unit and the second type of unit, is connected directly to the second type of unit and is thus connected indirectly to said control unit through the second type of unit and also through the first type of unit.

16. An ultrasonic diagnostic apparatus according to claim 15, wherein said plurality of transmitter-receiver units are connected directly to said control unit, said plurality of display units are connected directly to said plurality of transmitter-receiver units and are thus connected indirectly to said control unit through said plurality of transmitter-receiver units, and said plurality of operation units are connected directly to said plurality of display units and are thus connected indirectly to said control unit through said plurality of display units and said plurality of transmitter-receiver units.

17. An ultrasonic diagnostic apparatus according to claim 16, wherein at least one operation unit of said plurality of operation units and at least one display unit of said plurality of display units are connected directly to said control unit.

18. An ultrasonic diagnostic apparatus according to claim 15, wherein said plurality of transmitter-receiver units are connected directly to said control unit, said plurality of operation units are connected directly to said plurality of transmitter-receiver units and are thus connected indirectly to said control unit through said plurality of transmitter-receiver units, and said plurality of display units are connected directly to said plurality of operation units and are connected indirectly to said control unit through said plurality of operation units and said plurality of transmitter-receiver units.

19. A ultrasonic diagnostic apparatus according to claim 18, wherein at least one operation unit of said plurality of operation units and at least one display unit of said plurality of display units can be connected directly to said control unit.

20. A control unit for use with an ultrasonic diagnostic apparatus, said control unit configured to independently control:
a plurality of transmitter-receiver units, each transmitter-receiver unit comprising a probe connector, a transmitter-receiver, and a power supply;
a plurality of display units, each display unit comprising an image display and a power supply; and
a plurality of operation units, each operation unit comprising an operating part and a power supply.
